(19) United States
(12) Patent Application Publication
(10) Pub. No.: US 2013/0301003 A1
(43) Pub. Date: Nov. 14, 2013

(54) EYE EXAMINATION SYSTEM

(71) Applicants: Anthony Philip Wells, Roseneath (NZ); Nigel James Sharplin, Waikuku Beach (NZ)

(72) Inventors: Anthony Philip Wells, Roseneath (NZ); Nigel James Sharplin, Waikuku Beach (NZ)

(21) Appl. No.: 13/883,492

(22) PCT Filed: Nov. 7, 2011
(86) PCT No.: PCT/NZ2011/000236

§ 371 Date: Jul. 22, 2013

(30) Foreign Application Priority Data
Nov. 5, 2010 (NZ) 589038

(51) Int. Cl. A61B 3/00

(52) U.S. Cl. CPC A61B 3/0058; A61B 3/0091; A61B 3/0041
USPC 351/206; 351/246

(57) ABSTRACT

An eye examination system including a patient display visible by the patient during examination and an image capturing device arranged to take an image of the patient's eye during examination, the patient display being arranged to receive and display in real time the image for the patient to view during examination.

### FIELD OF THE INVENTION

The invention relates to an eye examination system and in particular to a slit lamp eye examination system.

### BACKGROUND OF THE INVENTION

A slit lamp is an instrument used in eye examinations for enabling diagnosis of a variety of eye conditions. The lamp consists of a high-intensity light source that can be focused to shine a thin sheet of light into the eye and is used in conjunction with a binocular microscope to provide a stereoscopic magnified view of the eye structure. A patient rests his or her chin and forehead on a support to steady the head. An ophthalmologist or optometrist can then use the lamp and binocular microscope to examine the patient's eye. Magnified images of the eye can be taken by a camera and displayed on a computer monitor for a more detailed review by the practitioner or further processing by an analytic system during or after the examination. During examination, the patient may be required to focus their eyesight on one or more objects so that the practitioner can shine the light on and examine a desired area/region of the eye.

It is an object of the present invention to provide an improved or at least alternative examination system enabling greater patient interaction.

### SUMMARY OF THE INVENTION

In a first aspect the invention may broadly be said to consist of an eye examination system including a patient display visible by the patient during examination and an image capturing device arranged to take an image of the patient's eye during examination, the patient display being arranged to receive and display in real time the image for the patient to view during examination.

Preferably the patient display is a micro display of screen area less than 100 cm², or less than 50 cm², or less than 20 cm² for example. More preferably the patient display is a micro LCD (liquid crystal display). Alternatively the patient display is a micro OLED (organic light emitting diode display).

Preferably the eye examination system comprises a slit lamp examination instrument having a microscope and an illumination unit coupled to a slit lamp base, and a support frame adjacent the illumination unit for supporting a patient's head to align the patient's eye with a light beam from the illumination unit and with the microscope for observation of the eye by a practitioner. Preferably the microscope is moveable relative to the frame. Preferably the illumination unit comprises an adjustment mechanism for altering one or more characteristics of the light beam.

Preferably the patient display is coupled to the support frame. Preferably the patient display is moveable relative to the frame. More preferably the patient display is coupled to the support frame via an articulating arm.

Preferably the system further comprises a dioptre adjustment mechanism for compensating for variations in patient focal length.

Preferably the patient display is powered by a power source associated with the illumination unit. Alternatively the patient display is powered via a separate power source.

Preferably the patient display is further arranged to display a focus object for the patient to focus on during examination. Preferably the focus object is overlayed on the image or the patient's eye.

Preferably the system further comprises a practitioner display arranged to receive image data and to display the image during examination for the practitioner and/or other observers to view.

Preferably the system further comprises a processing unit arranged to receive and process image data to at least aid in identifying one or more types of eye conditions from the image. Preferably the processing unit is arranged to manipulate the image data to highlight any identified eye conditions on the image displayed on either the practitioner display or the patient display or both.

Preferably the system comprises an input means associated with the processor and the practitioner display and/or patient display for receiving practitioner control commands to manipulate the output of the practitioner display or the patient display or both.

Preferably the input means can be used by the practitioner to control the location of a cursor displayed on the practitioner display and the patient display.

Preferably one or more targeting mechanisms are displayed on the patient display to provide specific means for referencing of aspects of the imaged region being examined, such as for example cross hairs and/or grid patterns.

Preferably the practitioner display has touch screen input capability.

Preferably the system further comprises a memory component arranged to receive and store image data from the camera during an examination session. The memory component may be local to the rest of the system or remote from the rest of the system.

Preferably the practitioner display or the patient display or both are arranged to display a series of images in sequence to provide a real time video display of the patient's eye during an examination session.

Preferably the system further comprises a microphone associated with memory to record patient and/or practitioner conversation during an examination session.

Any electrical connections between the camera, processor, practitioner display, input means, the memory component and the patient display may be wireless or via cable or any combination thereof.

Preferably the processing unit is arranged to identity one or more types of eye conditions.

Preferably the processing unit uses frequency and/or wavelength variation in the image to highlight different types of eye conditions.

In a second aspect the invention may broadly be said to consist of a method for carrying out an eye examination on a patient comprising:
viewing the patient's eye through a microscope,
simultaneously taking one or more magnified images of the patients eye, and
displaying the one or more images of the patient's eye on a micro display screen viewable by the patient during examination.

Preferably the image may be switched on or off or images other than the eye being scanned may be displayed on the micro display during the examination.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “examination” includes a surgical procedure on the eye (an image is displayed to the patient on the micro display during a surgical procedure of the eye).

### BRIEF DESCRIPTION OF THE DRAWINGS

A preferred embodiment of the invention will be described by way of example only and with reference to the drawings, in which:

FIG. 1 is a perspective view of a conventional slit lamp examination instrument,

FIG. 2 is a perspective view of a slit lamp examination instrument of the invention showing a camera with practitioner display,

FIG. 3 shows a patient being examined using a slit lamp examination instrument of the invention and patient display, and

FIG. 4 is a close up view of a patient being examined using a slit lamp examination instrument of the invention with another practitioner display.

### DETAILED DESCRIPTION

A preferred embodiment of the invention will be described with reference to the figures. The preferred embodiment outlines the use of the invention in a slit lamp examination instrument. The invention may be alternatively used in other eye examination instruments such as those used in the fields of optometry or ophthalmology. Eye examination instrument/system is therefore not intended to be limited to a specific field or area of practice. Examination systems can make use of the invention to improve patient interaction and understanding during examination.

In broad terms the invention therefore consists of an eye examination system including a patient display visible by the patient during examination and an image capturing device arranged during examination to take an image of the patient's eye being examined by a practitioner. The patient display is configured to receive data indicative of the image and to display the image for the patient to view during examination. The patient display is coupled to a support frame and may be moveable relative to the frame via an articulating arm for example for adjusting the position of the display relative to the patient. The display itself is preferably a micro display such as a micro LCD or OLED. The support frame and preferably in conjunction with the articulating arm allows the display to be positioned adjacent the patient for viewing of images of the patient's eye on the display. The display may be directly viewed by the patient or as will be described later the display may be adjacent an optical system, such as a periscope arrangement, through which the patient can view images of the examined eye.

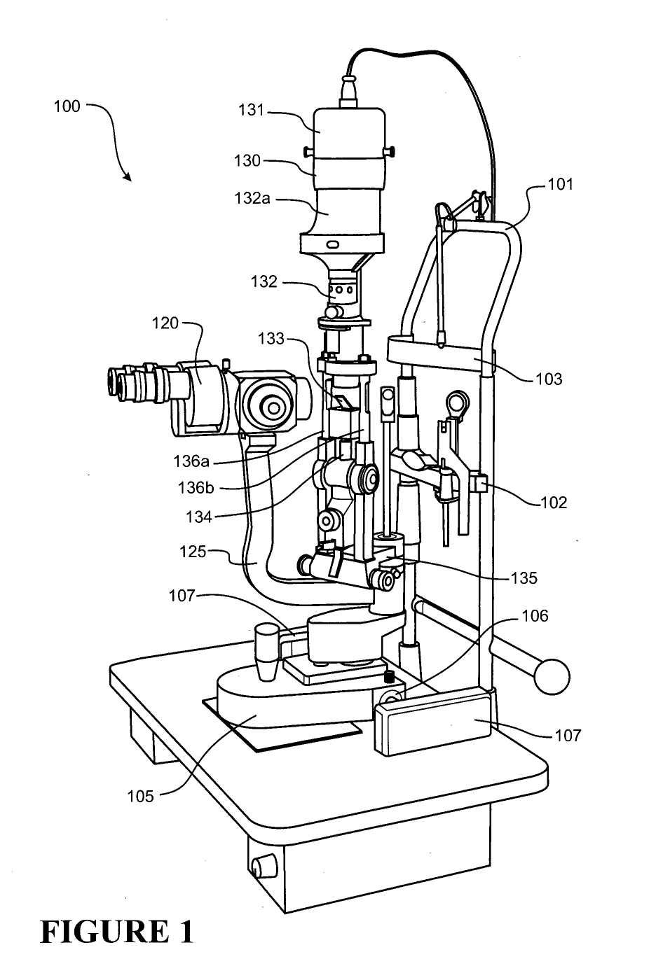

Referring to FIG. 1, a conventional slit lamp eye examination instrument 100 is shown comprising of a head rest frame 101 mounted on a lamp base 110. Chin and head supports 102 and 103 extend between parallel uprights 104 of head rest frame 11 respectively and ate vertically adjustable to permit proper alignment of a patient's eye. A carriage 105 is mounted on the slit lamp base 110 and is moveable over the surface of the base 110 to adjust the position of the vision components (described in more derail below) relative to the patient's eye. Carriage 105 moves laterally along rod 106 which in turn is accommodated at either end within a housing 107 in which the end of the rod can move by any suitable means. A vertical pivot pin 108 extends from the carriage 105 to pivotally support the microscope and illumination unit. Binocular microscope 120 is pivotally coupled to the base 110 via microscope support arm 125 and illumination unit 130 is pivotally coupled to the base 110 via illumination support arm 135 as shown.

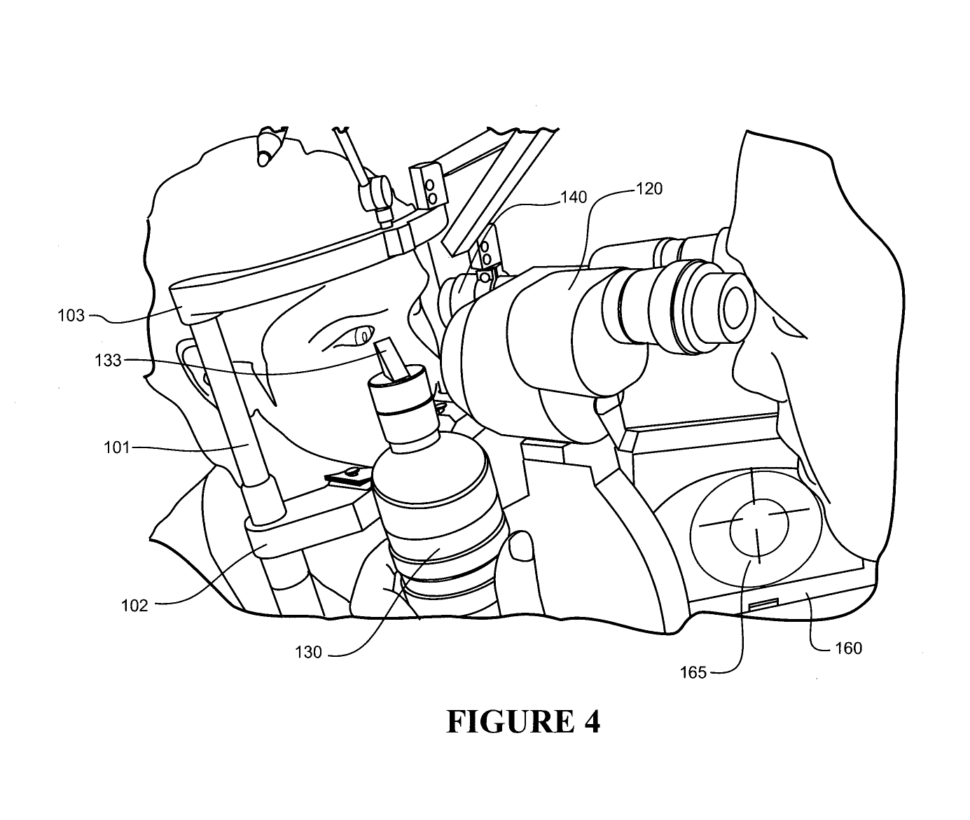

In a typical, standard slit lamp, of the type being described, original lamp unit 131 is mounted atop illumination column 132. Lamp unit 131 normally comprises an incandescent bulb (not shown) retained within a housing and a condensing lens (not shown). The components of the lamp unit 131 will not be described in more detail as any conventional unit may be used and the type of lamp unit is not intended to be the main subject of the invention. The bulb of lamp unit 131 directs a vertical beam of light through the condenser lens towards mirror 133 which is disposed at the bottom of the illumination column 132 on a post 134 to direct the light beam toward the eye of the patient. The mirror 133 is preferably oriented at substantially 45″ to the axial direction of column 132. Illumination column 132 is supported on the illumination arm 135 by a pair of spaced columns 136a/b. As understood in the art, upper column end 132a has a pair of cooperating members below the bulb and condensing lens which are controlled by a cam and control rod (not shown) to form a slit diaphragm tor adjusting the width of the slit of light used during examination. FIG. 4 for example shows a close up view of a patient being examined. A light beam from the illumination unit 131 is shown focused on the patient's right eye which is being examined by the practitioner through the binocular microscope 120.

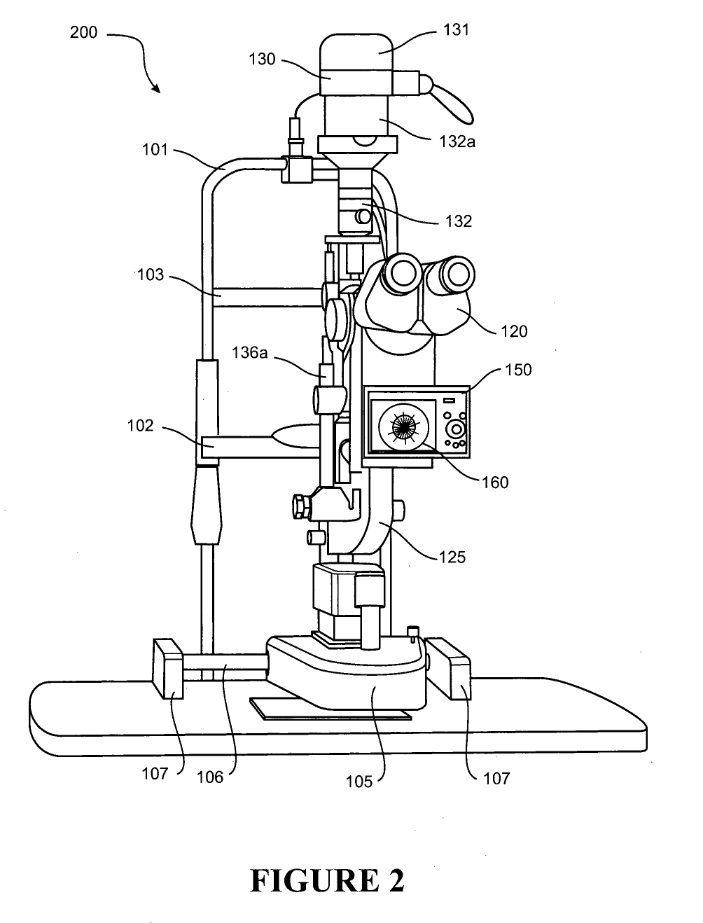

The above provides a description of the components of a typical slit lamp instrument, however, other known slit lamp instruments with differing components/mechanisms can be used instead and such known variations in the art are not intended to be excluded from the scope of the invention. The same reference numerals will be used on identical or similar components of the slit lamp instruments 200 of the invention shown in FIGS. 2-4.

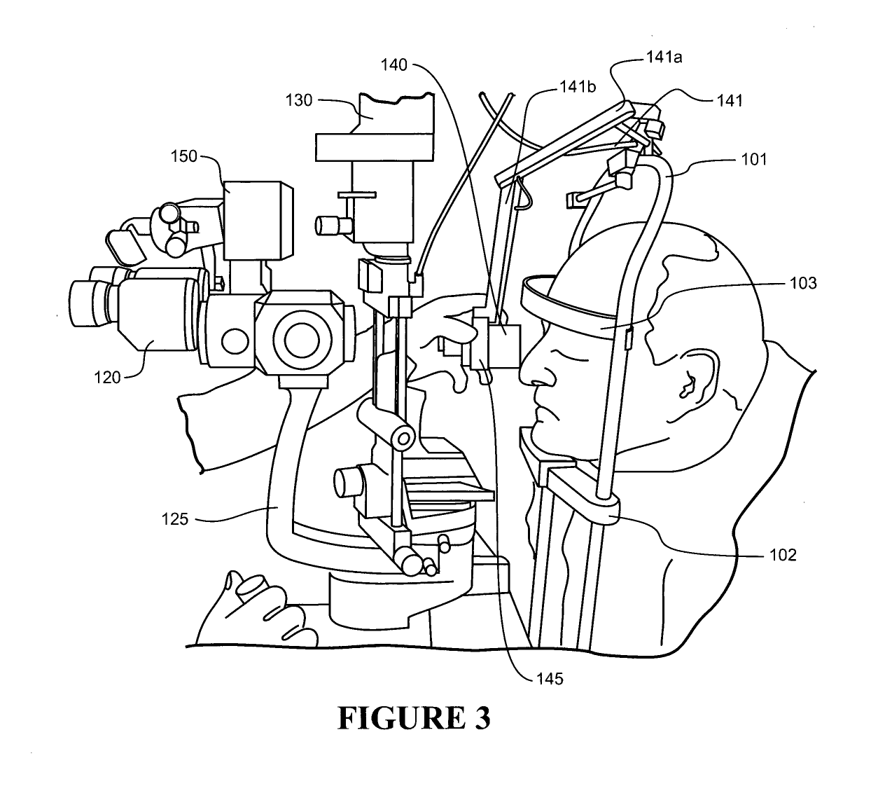

Referring now to FIG. 3, a patient display, preferably in the form of a micro display 140, depends from the head rest frame 101, and is used as a focus object for the patient and can be moved to a position tor maximum benefit during examination. The micro display 140 is arranged to display a focus object such as a red dot for instance which the patient may be directed to focus upon by the practitioner. The focus object provides a fixation target for the purpose of holding the patient's eye steady during examination. An articulating arm 141 extends from the head rest frame 101 for positioning the micro display 140 in front of the patient's eye and for moving the display 140 to an appropriate focus position as required by the practitioner. The arm 141 comprises two arm components 141a and 141b that are pivotally coupled to one another and to the head rest frame 101 to allow movement of the display 140 relative to the head rest fame 101 as shown in the figure. Arm 141a is pivotally coupled to the frame 101 such that it can rotate about two orthogonal axes to enable alignment of the display 140 with the patient's eye. Arm 141b is pivotally coupled to arm 141a such that it can rotate about one axis to alter the distance of the display 140 from the patient's eye. Any number of arm components necessary for providing the required degree of movement may be used. Furthermore, the arm components may be pivotally coupled to one another and/or to the head rest frame to enable rotation about any number of axes. The arm components may be manually moveable or may be associated with one or more motors for electronically adjusting the position of the display 140. The arm 141 may alternatively or in addition be flexible to enable the position of the display 140 to be altered appropriately. Alternatively in another embodiment the micro display is mountable to the head of the patient, for example by a wearable strap or headband.

The micro display 140 may comprise a screen of an area less than 100 cm², or less than 50 cm², or less than 20 cm² for example. The micro display 140 is preferably a micro LCD screen. Alternatively the display is a micro OLED. A dioptre adjustment mechanism 145 can also be employed by system 100 for the purpose of compensating for variations in patient focal length. The dipotre lens and adjustment mechanism 145 is closely associated with the micro display 140. The micro display 140 may be powered by the power source of the illumination unit 130 or any other suitable external power source.

The micro display 140 in the embodiment described needs to be positioned in front the patient's eye for viewing of images on the display by the patient. In alternative embodiments however, the display may not be positioned directly in front of the eye and an optical system adjacent the eye, such as mirrors forming a periscope-like arrangement may be used to help view the images on the display 140. The display 140 in such embodiments can be positioned above, below or to the side of the eye instead of directly in front with the optical system which in turn compensates for the positioning of the display 140.

The display 140, as well as providing the functionality of a focus object for the patient, is arranged to display images of the examined eye of the patient to enable the patient to see what the practitioner is observing during examination. This improves patient interaction during examination and can aid the patient in understanding any observations the practitioner may be articulating to the patient. The image of the patient's eye may be switched on or off or images other than the region being examined may be represented during the examination for improving patient interaction. These functions may be operated by the practitioner via a separate practitioner computer as will be described in more detail further. The image of the patient's eye preferably in real time may be used as the focus object for the patient during an examination. This has the advantage that as the focus object observed by the patient's moving eye is the eye moving image itself (when the patient's eye moves), this decreases the level of eye movement and better steadies the eye for improved examination. Alternatively as will be described later, a cursor or other pointer overlayed on the image of the patient's eye may be used as the focus object.

Referring now to FIGS. 2 and 3, the slit lamp eye examination system 100 further comprises an image capturing device 150 coupled adjacent the binocular microscope 120 to enable magnified images of the patient's eye(s) to be taken and analyzed in further detail by an optometrist/ophthalmologist. The image capturing device 150 is preferably a digital camera and may comprise a CCD chip, a CMOS chip, or any other suitable device.

The camera 150 is arranged to output digital image(s) to the micro display 140 such that a patient can view a magnified image of their eye in real time whilst focusing on the focus object also being displayed on the screen. The camera 150 may communicate with the micro display 140 wirelessly or via cable. The output image data from camera 150 can be sent directly to the micro display 140 or to a central processing unit which relays the image data onto the micro display 140. The central processing unit may process the image and overlay it with the focus object prior to sending the image to micro display 140 or the micro display 140 may overlay the object on the image itself. Presenting an image of the patient's eye during examination on micro display 140 provides visual feedback to the patient enabling the practitioner to make observations and communicate these clearly to the patient not only verbally but also visually in real time.

Referring now to FIGS. 2 and 4, in the preferred embodiment, the central processing unit is associated with a separate practitioner display unit 160 for also presenting the output images from camera 150 to the practitioner (and/or any other observers). The display 160 and processor are also associated with an input means. In the preferred embodiment the display 160 provides a touch activated input means but alternatively any other input means known in the art may be used. The processor is preferably arranged to communicate with the micro display 140 such that the practitioner can manipulate the image displayed on the micro display 140 for the patient through the input means and practitioner display unit 160. For instance, the practitioner display 160 may provide a cursor which can be moved on the display by the practitioner and viewed as a pointer device on the micro display 140 in the corresponding position against a background image of the patient's eye. Preferably the touch activated input means is configured such that the location of the finger or stylus used to move the cursor is offset from the cursor location to allow the object of interest to be observed un-obscured. Other mechanisms for highlighting important aspects of the image other than a cursor may be used. Furthermore, one or more targeting mechanisms may also be displayed on the practitioner 160 and/or patient displays 140 to provide specific means for referencing of position of aspects of the imaged region being examined. These may be cross hairs 165 as shown in FIG. 4 and/or grid patterns for instance. The system enables the practitioner to indicate to the patient an aspect of the eye using a cursor or other means and dynamically and in synchronization with the patient aligns this area in the central view on the patient 140 and/or practitioner 160 displays whereby the eye movement of the patient contributes to the process of alignment.

The central processing unit can be programmed or otherwise arranged to apply different image processing techniques on the images from camera 150 to identify (or to at least aid the practitioner in identifying) and/or highlight different types of eye conditions to be viewed by the practitioner on the display unit 160 and by the patient on the micro display 140. The central processing unit may use frequency or wavelength data associated with the digital data from each image to help identify types of eye conditions. Any image analysis technique known in the art may be used by the processing unit to determine an eye condition from image(s) captured by camera 150 during examination.

The practitioner display 160 or the patient display 140 or both are arranged to display a series of images in sequence as captured by the camera to provide a real time video display of the patient's eye during an examination session.

In the preferred embodiment, one or more memory devices are also associated with camera 150 to enable recording, storing and retrieval of video or still image data of eye examinations or other relevant data or information which supports the engagement of the patient. The memory device may be a local storage device or may be a remote data storage device which can be accessed by a computer online or via any other suitable network for example.

In the preferred embodiment a microphone is also provided in the system to record patient and/or practitioner communication and responses to visual images of the patient's eye. The microphone may be associated with the memory devices associated with the camera or any other separate memory device. The image data from camera 150 may be combined with audio data from the microphone to form a video of the examination session which can be stored on one or memory devices and viewed later. Other feedback mechanisms during the examination that benefit and or further enhance the patient's engagement or understanding of the image displayed can be used.

Any electrical connections between the camera, processor, practitioner display, input means, the microphone, the memory devices and the patient display may be wireless or via cable or any combination thereof.

The foregoing description of the invention includes preferred forms thereof. Modifications may be made thereto without departing from the scope of the invention.

1. An eye examination system including a patient display visible by the patient during examination and an image capturing device arranged to take an image of the patient's eye during examination, the patient display being arranged to receive and display in real time the image for the patient to view during examination.

2. An eye examination system comprising a slit lamp, a binocular microscope, a support frame for supporting a patient's head to align the patient's eye with a light beam from the slit lamp and with the microscope for observation of the eye by a practitioner, a digital image capturing device arranged to take images of a patient's eye during examination, and a patient display visible by the patient during examination and arranged to receive and display the images to a patient during examination.

3. An eye examination system according to claim 2 arranged to display a series of images in sequence on the patient display to provide a real time video display of the patient's eye during examination.

4. An eye examination system according to claim 2 wherein the patient display is arranged to also display a focus object for a patient during examination.

5. An eye examination system according to claim 2 wherein the patient display is arranged to display a focus object for a patient during examination, overlayed on an image of the patient's eye.

6. An eye examination system according to claim 2 wherein the patient display is arranged to display a real time image of the patient's eye as focus object for a patient during examination.

7. An eye examination system according to claim 2 arranged to display one or more targeting mechanisms on the patient display for referencing aspects of an imaged eye region being examined.

8. (canceled)

9. An eye examination system according to claim 2 wherein the patient display is a micro display of screen area less than 50 cm².

10. An eye examination system according to claim 2 wherein the patient display is a micro display of screen area less than 20 cm².

11.-12. (canceled)

13. An eye examination system according to claim 9 wherein the patient display is moveably coupled to the support frame.

14. (canceled)

15. An eye examination system according to claim 9 wherein the display is mountable to the head of the patient.

16. (canceled)

17. An eye examination system according to claim 2 comprising a practitioner display arranged to receive and display image or images of a patient's eye during examination to a practitioner and/or other observers.

18. An eye examination system according to claim 2 comprising a processing unit arranged to receive and process image data to at least aid in identifying one or more types of eye conditions from the image.

19. An eye examination system according to claim 18 comprising a processing unit arranged to receive and process image data to at least aid in identifying one or more types of eye conditions from the image and wherein the processing unit is arranged to manipulate image data to highlight any identified eye conditions on the image displayed on either a practitioner display or the patient display or both.

20. An eye examination system according to claim 18 wherein the system comprises an input means associated with the processing unit and a practitioner display and/or the patient display for receiving practitioner control commands to manipulate the output of the practitioner display or the patient display or both.

21. An eye examination system according to claim 20 wherein the input means can he used by a practitioner to control the location of a cursor displayed on the practitioner display and the patient display.

22. (canceled)

23. An eye examination system according to claim 18 wherein the processing unit is arranged to use frequency and/or wavelength variation in the image or images to highlight different types of eye conditions.

24. An eye examination system comprising a slit lamp, a binocular microscope, a support frame for supporting a patient's head to align a patient's eye with a light beam from the slit lamp and with the microscope for observation of the eye by a practitioner, an image capturing device arranged to image of a patient's eye during examination, and a patient display arranged to receive and display to a patient a series of images during examination and arranged to display a focus object to a patient during examination, overlayed on the image of the patient's eye.

25. An eye examination system according to claim 24 wherein the patient display is arranged to display a real time image of the patient's eye as focus object for a patient during examination.

26. A method for carrying out an eye examination on a patient comprising:
viewing the patient's eye through a microscope,
simultaneously taking one or more magnified images of the patients eye, and
displaying the one or more images of the patient's eye on a micro display screen viewable by the patient during examination.

27.-29. (canceled)
